(19) United States
(12) Patent Application Publication
(10) Pub. No.: US 2013/0172693 A1
(43) Pub. Date: Jul. 4, 2013

(54) DIAGNOSING SYSTEM FOR CONSCIOUSNESS LEVEL MEASUREMENT AND METHOD THEREOF

(71) Applicant: Penina Ohana Lubelchick, Tel Aviv-Yafo (IL)

(72) Inventor: Penina Ohana Lubelchick, Tel Aviv-Yafo (IL)

(21) Appl. No.: 13/820,949

(22) PCT Filed: Sep. 5, 2011
(86) PCT No.: PCT/IL11/00704

§ 371 Date: Mar. 5, 2013

(30) Foreign Application Priority Data
Sep. 7, 2010 (IL) 208055

(51) Int. Cl. A61B 5/00; G09B 19/00

(52) U.S. Cl. CPC A61B 5/48; G09B 19/00
USPC 600/301; 434/236; 705/14.66

(57) ABSTRACT

The present invention relates to a system and method for measuring behavioral change in human consciousness that is based on a personal code that allows diagnosis and determining the required method of treatment or decision. In particularly, the system is used for identifying the strengths and diagnostic discovery by objective feedback or response based on the patient's (i.e. subject) energy field, and not only by the subjectivity feedback arising due to the type of person the subject is. The system detects the subject's level of consciousness. The system can be used to identify the root elements of the subject's personality, so that through a short test could a therapist, the subject himself or even an organization that uses the system to receive results that will aid them to decide objectively or recommend them, of an efficient way of acting or operating.

### FIELD OF THE INVENTION

The present invention relates to the field of personality diagnosing system. More particularly, the invention relates to a universal system for measuring behavioral change in human consciousness that is based on a personal code that allows diagnosis and determining the required method of treatment or decision.

### BACKGROUND OF THE INVENTION

Consciousness is variously defined as subjective experience, or awareness, or wakefulness, or the executive control system of the mind. Some theorists—most of whom are physicists—have argued that classical physics is intrinsically incapable of explaining the holistic aspects of consciousness, but that quantum theory provides the missing ingredients. The most notable theories falling into this category include the Orch-OR theory formulated by Stuart Hameroff and Roger Penrose. Some of these quantum theories offer descriptions of phenomenal consciousness, as well as quantum interpretations of access consciousness.

The consciousness level of living organism is usually divided into four main levels: The first level is known as the instinctive level of consciousness, and it is defined as the lowest level of consciousness (mostly animals). The next level, the second level, is known as the emotional level. The third level is known as the logical thinking level where most of the people usually positioned, and the fourth level, the highest level, is known as the quantum thinking level. The ability to move up from the third level to the fourth level results in a change in the level of consciousness from a state of force to a state of power.

Dr. Valerie V. Hunt published a book “Infinite Mind: The Science of Human Vibrations of Consciousness”, which elucidates an energy field that permeates the body. Using Kirlian and enhanced computer photography, as well as new instrumentation to record the human energy field as vibrations (i.e., frequencies), Dr. Hunt's work goes well beyond current mind-body theories. In her continuing research into consciousness, Dr. Hunt explains that the goal is not to give you emotional peace and relaxation, or follow any particular philosophy, but to access the mind field, not the brain, in order to view everyday problems from a higher level of consciousness. This can be done as scientists who have redefined the mind have discovered vibratory frequency patterns that coexist with patterns of consciousness.

The Human Energy Field & Consciousness (as documented in the Bioenergy Research Laboratory at UCLA) discloses that the atomic electrical energy of the cells becomes the auric field. The field is both inside and outside of the body where it colors all sensory information that must pass through the field and skin before it gets to the sensory nerve endings. Because the field has a corona on the outside of the body it is in direct contact with all the energies of the environment, living, mineral, and celestial. This is the primary way humans interact with the world. The human field is a dynamic ever-changing series of patterns of frequencies that oscillate up to one million cycles per second making it the fastest and most elaborate of the body's electrical systems. Each person has a unique signature pattern of frequencies, his base reference, to which he returns when his field is not stimulated. This is a base reference from which his field responds to other fields. The mind-field is the highest level of memory, containing information about the physical body, thoughts, feeling states and past and present life experiences, particularly if memory is of profound experiences. Emotions connected with higher states of consciousness organize the mind-field and make it susceptible to new and threatening situations. This level of emotion is the deepest motivation of behavior.

Psychiatrist and consciousness researcher Dr. David R. Hawkins developed a “map” of the levels of human consciousness (also called the Scale of Consciousness) that uses a muscle-testing technique called Applied Kinesiology (AK) to document the nonlinear, spiritual realm. According to Hawkins, humans live at vastly different “levels” of consciousness. All these levels, along with the “truth level” of ANY true/false style inquiry, can be tested for truth and numerically “calibrated” through muscle testing, on a logarithmic scale of 1-1000. According to Hawkins, any person, concept, thought or object that calibrates at 200 (The level of Integrity) or above is positive (“power”); anything below 200 is negative (“force”).

According to Hawkins, the energy of a human thought is nonetheless absolutely measurable. A thought that emanates from the 100 level of consciousness will typically measure between log 10^{-800 million} to log 10^{-700 million} microwatts. On the other hand, a loving thought at the consciousness level of 500 measures approximately log 10^{-35 million} microwatts.

Briefly, each level of consciousness (LOC) coincides with determinable human behaviors and perceptions about life. Each level represents a corresponding attractor field of varying strength that exists beyond our three-dimensional reality. There's a critical point within each LOC from which its field gravitates (or entrains).

The numbers on the scale represent logarithmic calibrations (measurable vibratory frequencies on a scale which increases to the tenth power) of the levels of human consciousness and its corresponding level of reality. The numbers themselves are arbitrary; the significance lies in the relationship of one number (or level) to another. In the book “Power vs. Force: The Hidden Determinants of Human Behavior” by Dr. David R. Hawkins, there's an example for a hierarchy of levels of human consciousness (see FIG. 1). From low to high, the levels of consciousness are: shame, guilt, apathy, grief, fear, desire, anger, pride, courage, neutrality, willingness, acceptance, reason, love, joy, peace, enlightenment (e.g., Dr. Hawkins' scale is from 1 to 1000; a similar scale could be devised from 1 to 10 where 2 is courage and 5 is love).

While one can pop in and out of different levels at various times, usually there's a predominant “normal” state for each individual person. Hawkins defines these levels as a logarithmic scale, so there are far fewer people at the higher levels than at the lower ones. An increase from one level to another will result in enormous change in a person life. People can be ranked at these levels. Usually, within most people life, some parts of the life can be at different levels than others, but a person should be able to identify the current overall level.

According to the aforementioned researches and theories the level of consciousness can be defined as a range of frequencies, wherein each level is represented by specific frequency. Wherein, the higher the level of consciousness the higher is the frequencies. The value of each frequency is represented according to a logarithmic scale, thus the differences between adjacent frequencies values on the scale are relatively very high.

Progressing from one level to the next requires an enormous amount of energy. Personal growth experiences often occur in the form of a quantum leap—a strong and radical shift from one mindset to another. Without conscious effort or the help of others, many people likely just stay at their current level until some outside force comes into their life.

Going up even one level can be extremely hard; most people don't do so in their entire lives. A change in just one level can radically alter everything in someone's life. For example, people below the level of courage aren't likely to progress without external help. But, when someone hit the level of courage, all the past fears and false pride seems irrelevant.

In addition, today, most decisions of the people who need to make decisions, are results of job tests, human resources filtering, and other entities who need to define the personality type of a subject, all these are usually using techniques that are based on the subjective feedback from the subject, such as the answers of the subject to an introduced and predetermined set of questions. However, such techniques are very limited as they don't have the ability to provide an objective feedback from the subject.

Therefore, it is an object of the present invention to provide a system capable of obtaining the objective and the subjective feedback from a subject.

It is another object of the present invention to provide a system and method for aiding a person to improve the individual level of consciousness.

It is yet another object of the present invention to provide a system for detecting the personality type of a person.

It is still another object of the present invention to provide a system for providing recommendations to a person according to its personality type.

It is a further object of the present invention to provide a learning system which provides immediate treatment in real-time to a subject according to the subject's progress while using the system.

Other objects and advantages of the invention will become apparent as the description proceeds.

### SUMMARY OF THE INVENTION

The present invention relates to a system for detecting a universal consciousness personality type code of a subject, which comprises: a) an information processing unit suitable to execute software code for detecting one or more parameters related to the personality type of said subject, wherein said elements are detected by analyzing the subjective response of said subject, wherein said software code is used for determining a consciousness scale, wherein in each value on said scale is a logarithmic value which represents a frequency and for determining four personality parameters T, A, P and E for representing the individual personality type code of said subject, and for setting the ranges of each of said parameters on said consciousness scale, wherein the personality type code for each subject comprises three parameters among said four parameters, the first is a dominate element, the second is a stabilizer element and the third is a transformational element, wherein said dominate element is identified according to the subjective response of said subject; b) a data input device (e.g., a microphone or other voice sampling device) for collecting data representing voice parameters of said subject, wherein said voice parameters represent the subjective response of said subject; and c) voice spectral analysis means for extrapolating information from said subject voice in order to detect the dominate element of said subject.

The present invention further relates to a system for measuring the consciousness level of a subject, which comprises: a) data input device for collecting data representing the subjective response provided by said subject to an introduced set of questions, wherein said set of questions is used for determining the personality type of said subject; b) one or more sensing units for measuring the energy field of said subject while said subject response to each of said questions, wherein said measured energy field represents the objective response of said subject to each of said questions; c) memory unit for storing data representing all the relevant parameters of said subjective and objective response; d) information processing unit suitable to execute software code for detecting one or more parameters related to the personality type of said subject, wherein said elements are detected by analyzing the response of said subject to each question of said set of questions in correlations with the corresponding measured energy field, thereby determining the consciousness level of said subject; and e) data synchronization unit for synchronizing between the data representing each of the response provided by said subject to said set of questions and the corresponding measured energy field.

According an embodiment of the invention, the one or more sensing units are selected from the group consisting of: electromagnetic field sensors, Kirlian photography, Kinesiology based devices, Galvanic Skin. Response (GSR), ElectroMyoGraphy (EMG), ElectroEncephaloGraphy (EEG), or other instrumentation used to record the human energy field, etc., or combination thereof.

According to an embodiment of the invention the software code includes: a) a program for determining a consciousness scale, wherein in each value on said scale is a logarithmic value which represents a frequency; b) a program for determining four personality parameters Trust (T), Achievement (A), Pleasure (P) and Empire (E) for representing the individual personality type code of a subject, and for setting the ranges of each of said parameters on said consciousness scale, wherein the personality type code for each subject comprises three parameters among said four parameters, the first is a dominate element which represents the natural behavior of said subject, the second is a stabilizer element and the third is a transformational element, wherein said dominate element is identified according to the subjective response of said subject; c) a program for determining two pairs of said parameters, each of which comprises a dominate element and a stabilizer element, wherein the first pair of elements is E and A (or A and E) which represents of an extroverted personality characteristics, and the second pair of element is P and T (or T and P) which represents of an introverted personality characteristics; d) a program for determining three energy routes for categorizing each subject into one of said three energy routes by defining 12 basic personality types according to combinations of said four elements with said three energy routes, wherein the energy route of said subject is identified according to said subjective response of said subject and optionally other basic data related to said subject; and e) a program for identifying the stabilizer element of said subject according to the pair of elements in which said dominate element is found (i.e., E and A, or T and P), and for identifying the transformational element according the identified energy route of said subject and according to the extroverted or introverted personality characteristics of said subject.

According to an embodiment of the invention, the basic data related to the subject can be the date of birth, time of birth, family status, geographical location, etc. Additionally, the basic data is used for helping in estimating the hidden potential of the subject, thereby determining the highest consciousness level said subject may reach. In such case, the basic data can be obtained by testing said subject with common psychological test(s) and/or by introducing other relevant sets of questions (e.g., based on other known personality related theories or typologies).

According to an embodiment of the invention the system further comprises a decision support unit for providing recommendations to the subject according to the determined consciousness level of said subject. According to another embodiment the system further comprises a decision support unit for providing recommendations to the subject according to the identified transformational element and the energy route of said subject.

According to an embodiment of the invention, the system further comprises a display unit for introducing the set of questions to the subject, for displaying the determined consciousness level of said subject and for displaying the provided recommendations. Optionally, the 12 personality types can be presented in a circular form on said display.

According to an embodiment of the invention the system further comprises software code for presenting the 12 personality types on the nodes of a Metatron's cube for calculating the shortest route from the node representing the identified subject's personality to central node of said Metatron's cube, wherein said shortest route represents the minimum effort required from said subject in order to increase the level of consciousness of said subject. Calculations of the shortest route are based on the “critical point” of the Chaos theory, which identifies the parameter that will cause the maximal effect while providing the minimal effort. The transformational element refers herein to this “critical point” or shortest route.

According to an embodiment of the present invention, the system further comprises at least one database for storing the categorized subjects according to the set of questions, wherein said at least one database is used for matching between at least two subjects having at least the same dominate elements.

According to an embodiment of the present invention, the system further comprises treatment database for storing data related to different way of treatments to the subjects, wherein said treatment database is either located locally in relation to said system or remotely. Optionally, the remotely located treatment database can be accessed via the Internet or other network protocols. The provided treatment to the subject via the treatment database can be provided in real-time while said subjected is being tested by said system, thereby allowing said system to provide an adaptive treatment to said subject according to the progress of said subject during said test.

According to an embodiment of the present invention, the system further comprises voice capturing and analyzing means for detecting the dominate element in the personality consciousness type code of the subject.

The present invention further relates to a method for detecting a universal consciousness personality type code of a subject, comprising: a) determining a consciousness scale, wherein in each value on said scale is a logarithmic value representing a frequency; b) determining four elements T, A, P and E for representing the individual personality code of said subject, and setting the ranges of each of said elements on said consciousness scale, wherein the individual code for each subject comprises three elements among said four elements, the first is a dominate element, the second is a stabilizer element and the third is a transformational element; c) determining two pairs, each of which comprises a dominate element and a stabilizer element, wherein the first pair of elements is E and A (or A and E) which represents of an extroverted personality characteristics and the second pair of element is P and T (or T and P) which represents of an introverted personality characteristics; determining three energy routes for categorizing each subject into one of said three energy routes by defining 12 basic personality types according to combinations of said four elements with said three energy routes; and d) detecting the dominate element and the energy route of said subject by obtaining the subjective response of said subject to one or more tests, wherein said dominate element and said energy route of said subject are used for categorizing said subject into one of said 12 basic personality types.

According to an embodiment of the present invention, the one or more tests are selected from the group consisting of: a set of questions introduced to the subject wherein the answer of said subject to each of said question represents the subjective response of said subject, a voice spectral analysis of the subject's voice or combination thereof.

The present invention further relates to a method for measuring the consciousness level of the subject, by performing the steps of: a) measuring the energy field of said subject by one or more sensing units, wherein said measured energy field represent the objective response of said subject to each of the introduced questions; b) comparing the subjective response of said subject in correlations with the objective response of said subject, thereby determining the consciousness level of said subject wherein while the difference between said subjective response and said correlated objective response is relatively high, then the consciousness level of said subject is relatively low, and vice versa; c) identifying the stabilizer element of said subject according to the pair of elements in which said dominate element found; and d) identifying the transformational element according the detected energy route of said subject and according to the extroverted or introverted personality characteristics of said subject.

According to some embodiments of the present invention, the method further comprises providing a decision support unit for providing recommendations to the subject according to measured consciousness level.

According to an embodiment of the present invention, the method further comprises providing treatments or treatment suggestions for the subject according to the identified transformational element of said subject or according to difference between the objective and the subjective responses of said subject. For example, the treatment can be obtained by providing music with adjustable frequencies (by using frequency generated equipment), or any other treatment which may increase the frequency of the subject and thereby increase its level of consciousness.

According to an embodiment of the present invention, the method may further comprises obtaining basic data related to the subject for helping in estimating the hidden potential of said subject, thereby determining the highest consciousness level said subject may reach. Such basic data can be obtained by psychological test(s) and/or other relevant sets of questions.

According to an embodiment of the present invention, the 12 basic personality types are visually shown on a circular TAPE code diagram. Alternatively, the 12 personality types can be presented on the nodes of a Merkava model or Metatron's cube for calculating the shortest route from the node representing the identified subject's personality to central node of said Merkava model or said Metatron's cube, wherein said shortest route represents the minimum effort required from said subject in order to increase the level of consciousness of said subject.

According to some embodiments of the present invention, the method further comprises matching between at least two subjects having at least the same dominate elements, by providing at least one database for storing the categorized subjects. The matching can be done by identifying at least one of the following four elements Mo, Fr, Im, Pr, wherein all of said four elements are directly related to each other, while a function of them enhances or increases the chances in succeeding with “closing” a transaction. The matching is based on the assumption that as much as the dominant elements of the subjects are similar, then said subjects are better matched. For example, the matching can be used for gathering persons for team work, matching between salesman and client(s), husband and wife, teacher and student, etc.

According to an embodiment of the present invention, the matching may comprise a clients' channel, a marketing channel, and/or an agents' channel. The clients channel comprises the steps of: a) building one or more sets of questions for identifying the dominant element of each client; b) providing a set of a questions to each client, via any applicable communication method, and according to response of said client to said set of question, categorizing each client profile according to at least the dominant element of each client; and c) storing each categorized client in a clients' database. The marketing channel comprises the steps of: a) analyzing the way of introducing the same product according to each defined personality type; b) generating targeted messages for each personality type; and c) matching between generated targeted messages and the clients according to their stored categorized profile and delivering said generated targeted messages to said clients. The agents channel comprising the steps of: a) providing a set of questions to each agents for categorizing each agent according to the 12 personality TAPE types; b) storing each categorized agent in an agents' database; and c) matching between profiles of agents stored in said agents' database and clients stored in the clients database having the same dominate personality type, thereby increasing the chances for “closing” a transaction.

According to an embodiment of the present invention, the method further comprises displaying the subject's personality according to the identified elements.

According to an embodiment of the present invention, the method comprises identifying an element which drives or motivates the subject to perform an action, wherein said element is derived from the dominant element of said client.

### BRIEF DESCRIPTION OF THE DRAWINGS

In the drawings:

FIG. 1 is a table showing an example of scaling the levels of consciousness;

FIG. 2 schematically illustrates a level of consciousness graph in correlation with TAPE types, according to an embodiment of the present invention;

FIG. 3 schematically illustrates a TAPE code diagram in a circle form, according to an embodiment of the invention;

FIG. 4A schematically illustrates a TAPE code diagram presented in a Metatron's cube model, according to an embodiment of the invention;

FIG. 4B schematically illustrates the transformation from one dimension to another using the Metatron's cube model;

FIG. 5 schematically illustrates a block diagram of a decision making support system for consciousness level measurement, according to an embodiment of the invention;

FIG. 6 schematically illustrates the system of FIG. 5 provided with a treatment database, according to one embodiment of the invention; and

FIG. 7 schematically illustrates a matching system between clients and agents, according to another embodiment of the invention.

### DETAILED DESCRIPTION OF PREFERRED EMBODIMENTS

The present invention relates to a universal system for measuring behavioral change in human consciousness that is based on a personal code that allows diagnosis and determining the required method of treatment or decision. Further information can be found in the book “law of power” by the inventor of the present invention. In particularly, the system is used for identifying the strengths and diagnostic discovery by objective feedback or response based on the patient's (i.e., subject) energy field, and not only by the subjectivity feedback arising due to the type of person the subject is. The system detects the subject's level of consciousness. The system is discriminator level of universal consciousness of their characters, no matter what type of typology is used, the system can be used to identify the root elements of the subject's personality, so that through a short test could a therapist, the patient himself or even an organization that uses the system to receive results that will aid them to decide objectively or recommend them, of an efficient way of acting or operating.

The method of the present invention is based on the common assumption that the human personality operates like a series of psychological functions, and that a human knows things that he does not always aware of them, but they are expressed in the responses of his body.

While different combinations of such functions create different ways to receive and to produce information. Humans always strive for happiness and the joy of creation, to be balanced in the universe. It is well known that there are different personality types of people which drive or act by different types of energy, such as businessmen, inventors, leaders, employee, etc. Usually these energies are genetics. According to an embodiment of the invention, the energy that motivates or drive people is categorize into one of three main energy types, which are defined as Dominant energy people (D), Constant energy people (P) or Varied energy people (N). Although each person is unique and form different combinations of these types, most people have one dominant personality and energy type that characterizes them most accurately.

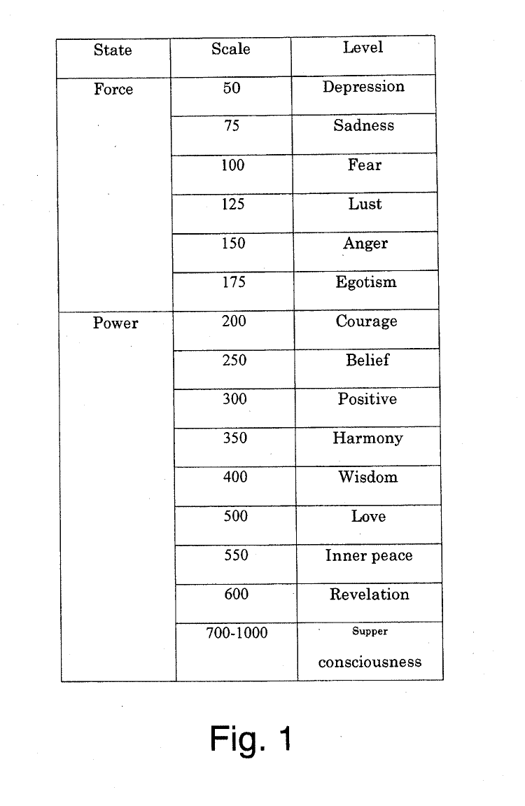

Essentially a person aspires to play the game of life in maximum of security, awareness, control, pleasure and success. This is the goal of most people in their life; when one achieves this goal, the person “moves” with a great energy, think fast, achieves success in reality, creates reality, shares with others, full of joy, full of laughter, becomes lightness, achieves goals, etc., all of these great features become possible when the person comes to a relatively higher level of consciousness (i.e., move up from a LOC in the range of force to a LOC in the range of power, as shown in FIG. 1). However, many people live their life on an “automatic pilot” mode where they make their decision from their subjective self, which is usually located in the range of force in the LOC. Therefore one of the objects of the system and method of the present invention is to aid people to make decision from a relatively higher LOC.

FIG. 1 shows a table in which the levels of consciousness are represented by logarithmic figures based on the scale of Dr. Hawkins, i.e., it means 10 to the power of each figure (e.g., level 200 is not the absolute number 200. Thus, 200 on the scale, does not mean twice the magnitude of 100). Therefore, a small increment by a few points represents a huge increase in magnitude. For example, let's look at the values 175 and 200. Even though it's just difference of +25 in pts on the scale, according to the logarithmic terms this means that it will result in enormous improvements in his well being and state of life.

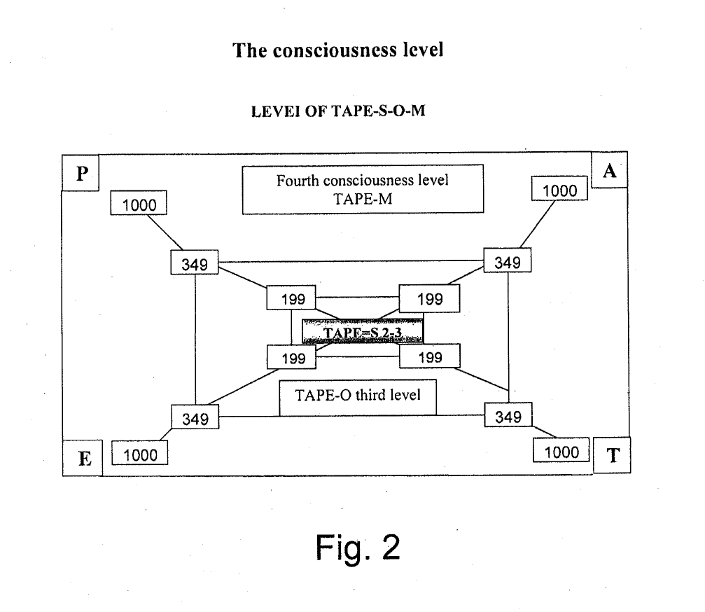

According to an embodiment of the invention, the suggested method is based on the assumption that there are four main elements which represent the individual personality type code of a subject/a person (i.e., this code may refer to as the root or DNA-like personality code). The elements are defined as Trust, Achievement, Pleasure and Empire (i.e., the TAPE code of a person) as shown with respect to FIG. 2. The terms “personality type” or “personality type code” refers herein to the TAPE code of a person. Following is a more detailed description of the four elements of the TAPE code:

T—Represents the level of Trust of a person which can be positioned on the level of consciousness axis between Fear to Trust;
A—Represents the level of Achievement of a person which can be positioned on the level of consciousness axis between the inability of doing (i.e., depression) up to the desire for achievement;
P—Represents the level of Pleasure of a person which can be positioned on the level of consciousness axis between Sadness to Pleasure (joy);
E—Represents the level of Empire of a person which can be positioned on the level of consciousness axis between Egoism to Altruism (mastery or control).

By decoding the encrypted data of the subject's TAPE code, one can diagnose the strengths and weaknesses of the subject, the hidden potential inherent in the subject, and to obtain the optimal way to realize this hidden potential.

The novelty of the TAPE model is that no matter what criteria or diagnostic typology of personal identification is used, the TAPE model can decode and show the strengths and weaknesses of the subject as well as the shortest way to treat the subject (i.e., to move up in the level of consciousness scale). According to an embodiment of the invention, the level of consciousness scale is divided into three consciousness ranges (0-199, 200-349 and 350-1000) which are defined here as TAPE-S, TAPE-O and TAPE-M.

TAPE-S—This is a subjective self type (i.e., the “automatic pilot” type). Every human is the total resulting from his thoughts, feelings, his life events and his memories. Usually represent people who act “automatically” by their subjective self and their decisions are taken from there. This represents the lowest ranges in the LOC scale (0-199). Many people usually located in this range on the scale. This level can be defined as the instinct based level (i.e., “attack and run”).

TAPE-O—This is an objective type (i.e., the “self pilot” type). A balance type of person, act by his own intelligence and imagination, has the ability to distinguish between true and false, between main things to subordinate, etc. This represents people in the middle ranges in the LOC scale (200-349). This level can be defined as the logical thinking level (i.e., “think before attack”).

TAPE-M—This is the complete personality type (i.e., the higher self). A person, that works with the past and with a vision to the future, and therefore can make correct decisions in a wise way. Someone who acts with maximum attention in the present and by that understands and remembers his life experiences and produces from them a pure study. This represents people in the highest ranges in the LOC scale (350-1000)—the quantum thinking level (i.e., the fourth dimension).

The following table (1) summarizes the three TAPE ranges with respect to the level of consciousness:

TABLE 1

| | TAPE-S | TAPE-O | TAPE-M |
|---|---|---|---|
| Who rules | Automatic Pilot | Self Pilot | Higher Self |
| Mind level | Beta | Alpha | Theta and |
| | | | Delta |
| Behavior | Survival, act | Balanced, | |
| | by fear, mostly | connected | |
| | egoism | to reality | |
| Level of | 0-199 | 200-349 | 350-1000 |
| Consciousness | 2^{nd}-3^{rd} level | 3^{rd} level | 4^{th} level |
| World view | Frighten | Optimist | Harmony, joy |

According to an embodiment of the invention, each person has a TAPE-S level and a TAPE-O level. Preferably, the TAPE-S level of a person is determined according to the person's subjective answers (i.e., responses) to a set of questions, and the TAPE-O level of a person is determined according to the energy field or physiological vibrates of the person's body which measured in accordance to each answer the person provide (i.e., to each person's response).

According to an embodiment of the present invention, in order to allow a person to be in control (i.e., to move up and to act in the levels of TAPE-M), it is required to reduce the distance (i.e., the delta, the difference on the LOC scale) between TAPE-O and TAPE-S of that person to minimum, preferably to zero. By doing so, one can get himself closer to TAPE-M type. Preferably, the way to reduce this delta depends on the structure (or level) of the elements of each individual (i.e., the four T, A, P, E basic elements that characterized each individual person—the TAPE).

According to an embodiment of the invention, in order to increase the LOC of a person, the following element of the subject person should be identified:
The dominant element of a person—which is usually the prominent element in the human behavior. This element is usually very easy to detect. This element will be the “glasses” of the person in all types of TAPE for that person;
The second element of a person—is the stabilizing element (i.e., the stabilizer). The second element is depended on the dominant element; and
The third element—is the hidden element of the person. This element will be the transformational element. This is the element that required doing the minimum effort from the person in order to allow that person to move up from the current LOC to a higher LOC. This can be done as the relations between the elements (dominate, stabilizer and transformational) is such that a relatively slight change in the transformational element (i.e., the “critical point”) will result in the largest change in the other two elements, mainly in the dominant element.

According to an embodiment of the invention, the minimum effort refers to the type of treatment or to the relatively minimal change in the personality aspect that a person should do in order to cause to a relatively larger change in the dominant element of that person. Usually, the ratios between the elements of a person are determined as follows: the dominate element reflects 50% of the personally, the stabilizer element reflects 30% and the transformational element reflects 20%. For example, if a person dominate element is detected as “T” (with constant energy type P), the stabilizer element is then “P” and the transformational element is accordingly identified as “A”, then a relatively small change in the value of “A” will significantly increase the value of “T” as it constitutes 50% of the personality.

These elements are different from one person to another, and they draw the lines that affect a person's energy, stabilization or balance. As aforementioned defined, most people are on the three routes of energy: variable (N), constant (P) and dominant (D). Once the dominate element (from the four T, A, P and E elements) and the energy route (among the three defined energy routes) are detected, we know what a track—a code element, according to the TAPE coed and what formula we need to use, in order to reduce the delta between TAPE-S and TAPE-O with relatively minimum effort from the person. Preferably, the dominate element and the energy route are determined according analysis of to the person's response to a set of questions and optionally, other data related to the subject (such as date of birth, time of birth, geographical location, etc). The set of questions are built in such a way that each person can be distinguish and categorized according to the dominate TAPE element type and the energy route, as are known to a person skilled in the art.

The trajectory of the transformational element is the energy of the optimal route to each particular person. This is the code to begin cleaning the roadblocks and fears that actually created the separation and the distance from the higher self. The more we are loaded with energy we may clean up more from the “extra armor” that hides our real personality, and return control, from the “automatic pilot” (i.e., TAPE-S), to my own self to TAPE-M.

In the prior art, most decisions of the people who make decisions, job tests, human resources filtering, and the like, use equipments which their feedback from the subject was based on its TAPE-S only (i.e., on the subjective feedback from the subject). Usually, such equipments do not reveal the hidden potential of the subject or his real skills. In contrary, the system of the present invention provides a device capable of receiving feedback from the TAPE-O type and the TAPE-S type of the subject and to calculate the distance between them. The distance or difference between these TAPE types can indicate in what level the subject is really positioned from a consciousness point of view (e.g., these reflects the abilities of the subject that are hidden within him, and might revealed or expressed whenever the subject will feel comfortable in a suitable job or environment).

Usually, TAPE-S is determined by providing the subject only with a set of questions on which the subject needs to answer. According to the subject's responses the subject's personality type is determined. However, as aforementioned, the subject's responses are based on his subjective act. The system of the present invention comprises one or more sensing units which measure the subject's energy field as well as optionally other physiological response while the subject answers to the set of questions. Preferably, each measured response of the subject's body is correlated with a specific answer. TAPE-O is determined according the measured response of the subject's body (by the sensing units), while TAPE-S is determined according to the subjective response of the subject to the set of questions (in this case the subjective response are represented by the subject's answers). The physiologic sensing means can be any measuring unit capable of sensing or measuring human energy field, Kinesiology based measuring devices, and the like. In addition, other common types of human body sensing means can also be used.

According to an embodiment of the invention, the system compares the measured response of the subject's body (i.e., the objective response) with the subjective response of the subject (i.e., it compares TAPE-O with TAPE-S) and accordingly it calculates the delta between them (i.e., it calculates “how far” is TAPE-O from TAPE-S of the subject).

According to an embodiment of the invention, in order to identify the personal TAPE code of each subject, a TAPE code diagram or “map” should be used through which the three individual code of a person can be detected.

The terms “extroverted” or “extroverted personality” refers herein to a person, who usually motivates with respect to an external energy source, e.g., a person two easily interacts with group of people, a friendly character.

The terms “introverted” or “introverted personality” refers herein to a person, who usually motivates with respect to an internal energy source, e.g., a person who prefers privacy or one on one contact with other people and less prefer contact with a group of people.

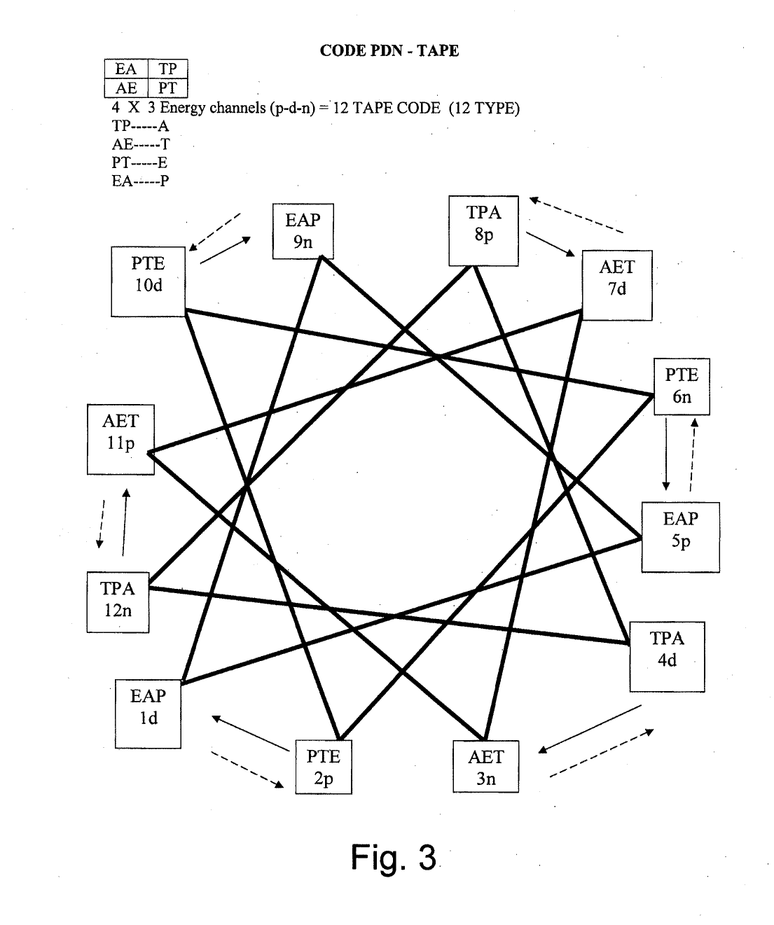

FIG. 3 schematically illustrates an example for such TAPE code diagram, according to an embodiment of the invention. The diagram shows possible directions of energy routes (wherein an extroverted personality characteristic is shown by the dotted arrows in the counterclockwise direction, and an introverted personality characteristic is shown by the non-dotted arrows in the clockwise direction) according to the three main types of human energy (wherein “p” represents people having constant energy, “d” represents people having dominant energy and “n” represents people having varied energy). The TAPE code is represented by a circle form in which the TAPE code is formed three times in a circular manner, which gives a total of 12 options or combinations for defining 12 different basic personality types of individual subject. Each of the four elements can connect with each of the energy types p, d and n (i.e., 12 options).

The following Table (2) summarizes the 12 personality types as defined by the TAPE code with respect to the diagram of FIG. 3. Table (2) shows the three elements of each personality type, the energy channel and the corresponding extroverted or introverted character of each type.

TABLE 2

| The | | | Energy | | Stabi- | Transfor- |
|---|---|---|---|---|---|---|
| code | | Extroverted/ | channels | Dominate | lizer | mational |
| No. | Type | Introverted | p-d-n | element | element | element |
| 1 | EAP | Extrovert | d | E | A | P |
| 2 | PTE | Introverted | p | P | T | E |
| 3 | AET | Extrovert | n | A | E | T |
| 4 | TPA | Introverted | d | T | P | A |
| 5 | EAP | Extrovert | p | E | A | P |
| 6 | PTE | Introverted | n | P | T | E |
| 7 | AET | Extrovert | d | A | E | T |
| 8 | TPA | Introverted | p | T | P | A |
| 9 | EAP | Extrovert | n | E | A | P |
| 10 | PTE | Introverted | d | P | T | E |
| 11 | AET | Extrovert | p | A | E | T |
| 12 | TPA | Introverted | n | T | T | A |

Finding the dominant element of the subject can be easily detected by performing relatively simple tests, such as using the set of questions.

Finding the second element, the stabilizer element, is based on the assumption that the second element is dependent on the dominant element. Therefore there can be only a limited combination of a connection between the dominant and the stabilizer elements, which, according to the TAPE code diagram, can only be the element couples (E and A, or A and E) or (P and T, or T and P). E and A represent an extroverted personality, while P and T represent introverted personality.

Finding the third element—the transformation element—depends on the extroverted energy or the introverted energy of the subject. In case the subject is an extroverted type, then the energy moving route is in a counterclockwise direction (i.e., E and A, or A and E) along the TAPE code diagram. On the contrary, in case the subject is an introverted type, then the energy moving route should be in a clockwise direction (i.e., P and T, or T and P) along the TAPE code diagram.

According to an embodiment of the invention, the decoding is done as follow:
1. Detection of the dominant element and the energy direction, by giving a set of questions to the subject and processing the subject's answers to them;
2. Once the dominant element and the energy direction are detected, it is immediately possible to identify the second element, the stabilizer, according to the couples E and A (or A and E), or P and T (or T and P); and
3. Identifying the transformational element. The identification of the transformational element is done according to the type of person whether the person is extroverted or introverted. This type provides the clockwise or counterclockwise movement in the TAPE code diagram. The transformational element is defined according to the identified energy route of the subject and according to the direction detected based on the personality characteristics i.e., extraverted type (A and E) will move counterclockwise and the introverted type (P and T) will move in clockwise direction. When we follow this rule we find that the transformational element of one type will be the dominant element of the next type on the circle-like diagram of FIG. 3, according to the direction described above.

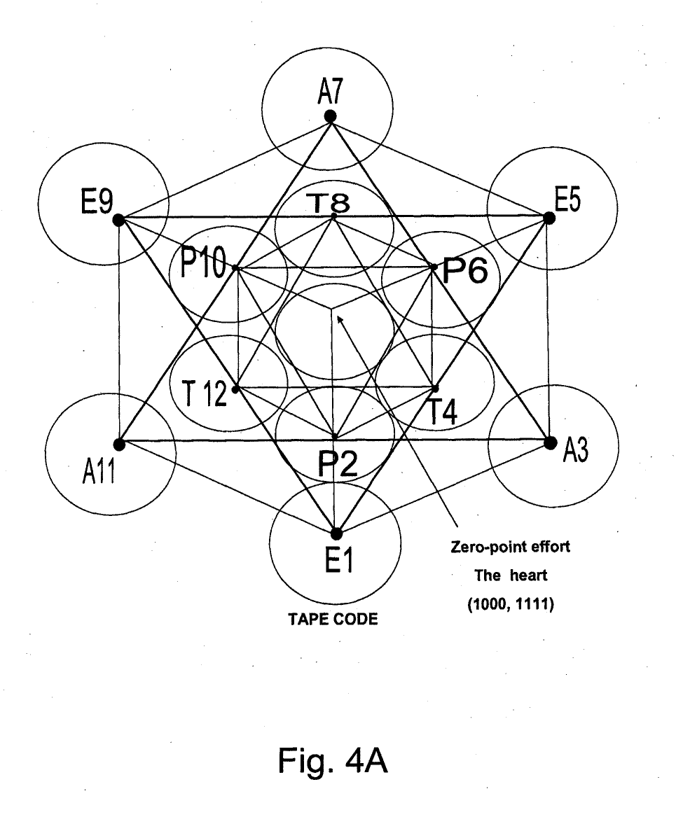

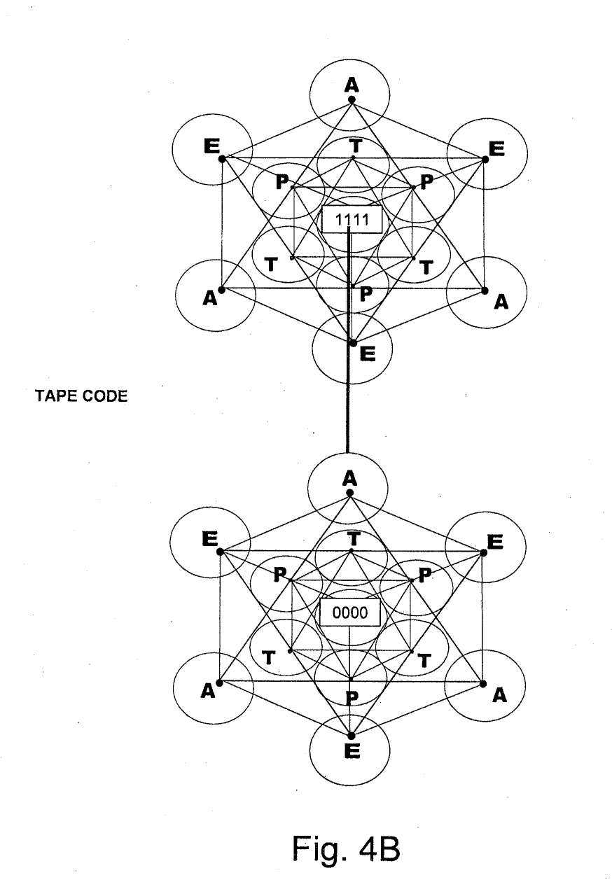

According to an embodiment of the invention, the TAPE code diagram can be presented in form of a Merkava model (i.e., a form of 3D star of David) or in form of a Metatron's cube model as shown with respect to FIGS. 4A and 4B. The Metatron's cube represent a three dimensional module of two Stars of David, which represents a box within a box or a cube nested within a cube. Each circle represents a node, wherein a three pairs of the elements A and E represent nodes on the external cube and a three pairs of the elements P and T represent nodes on the internal cube. Two other nodes 0000 and 1111 represent the “heart” of the Metatron's cube (these two nodes can be seen on FIG. 4B as they appear in different dimensions, and they demonstrate the transfer or the bridge between one dimensions to the other). A maximum balanced TAPE code of a person is obtained when the TAPE code is located on these “heart” nodes (i.e., a person having a relatively high LOC). Therefore, one of the objects of the present invention, is to identify (e.g., this can be calculated by dedicated mathematical model installed within the software used by the system of the present invention) where a specific person is positioned on the Metatron's cube as well as the energy direction (i.e., the location of the detected TAPE code of that specific person and the direction required to move toward one of the “heart” nodes), and what type of treatment that person needs in order to bring him towards the “heart” nodes (i.e., to increase the LOC of that person).

According to an embodiment of the invention, after the subject has been diagnosed by the system of the present invention (i.e., the three individual TAPE code of the subject were detected—the dominant, the stabilizer and the transformer), the subject's TAPE code can be positioned on the nodes of the Metatron's cube and accordingly the shortest route and its direction to one of the “heart” node can be calculated (e.g., by using Boolean algebra functions, wherein each node is represented by 4 digits combination of the two values “0” and “1”, Gunn square, Fibonacci numbers, etc.). The shortest route reflects the minimum effort a person should do (i.e., treat or “fix”) in order to move up to a higher LOC. The minimum effort is represented by the detected transformation element of that person (i.e., this is the element that should be treated or “fix” in order to move up to a higher LOC. The treatment can be any suitable way of treatment, such as psychological, alternative medicine, coaching, Vibrational medicine or Vibrational Therapy, etc).

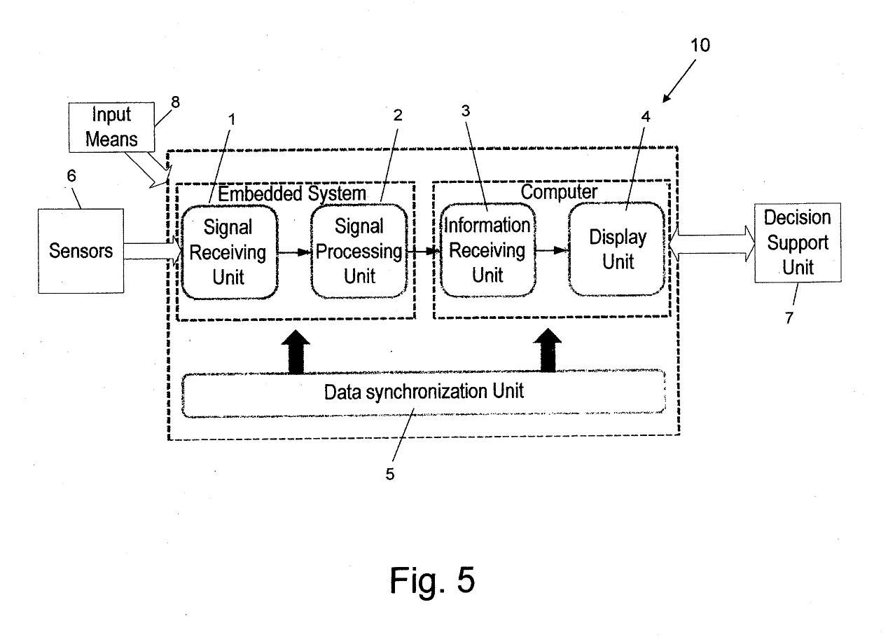

Referring now to FIG. 5, a system 10 for detecting personality type and LOC is shown in a block diagram form, according to an embodiment of the invention. System 10 comprises a Signal Receiving Unit (SRU) 1, Signal Processing Unit (SPU) 2, Information Processing Unit (IPU) 3, Display Unit (DU) 4, Data Synchronization Unit (DSU) 5, sensing means 6, input means 8 and optional Decision Support Unit 7. The sensing means 6 are used for measuring the subject's energy field or other physiological body vibrations which are “translated” by the Information Processing Unit 3 to the subject's objective consciousness level (represented by TAPE-O).

System 10 operates as follow: One or more one sensing units 6 are set to sense or measure the subject's body vibrations or energy field. Optionally, a sensing unit can used to measure other physiological body response of the subject, such as Biosensor to measure the Biofeedback Data, which is the Electro-Dermal Activity and Electrical Conductivity of the skin of the subject and it's Temperature (e.g., the subject's hand).

Display Unit 4 introduces pre-defined set of questions (e.g., TAPE based questionnaire for categorizing the subject personality) to the subject, treatment suggestions and decisions. Input means 8 are one or more data input devices which are used to receive the subject's response to the introduced questions (i.e., the subjective response—subject's TAPE-S).

During the subject's answers to the questions, signal receiving unit (SRU) 1 receives input signals from sensing unit 6 as electromagnetic wave variations (this received signals will be processed and translated into the TAPE-O representation of the subject—the objective response). SRU 1 amplifies the received analog input signal, digitizes that signal and converts it into a digital signal. Signal Processing Unit (SPU) 2 receives the converted digital signal, processes the received digital signal and outputs data, which represent the subject's physiological body response (e.g., energy field or other body vibrations), to the Information Processing Unit (IPU) 3.

IPU 3 receives the data from SPU 2, analyzes the received information and makes mathematical computations and analysis. The IPU 3 also sends data to Display Unit 4 for showing the subject or operator of system 10 the mathematical analysis results and optionally the decision support recommendations from Decision Support Unit 7. Data Synchronization Unit 5 synchronizes the received signals from sensing unit 6 and the data received from the input means 8 (i.e., in order to compare between the subject subjective response to the set of questions and the subject's body vibrations).

Sensing unit 6 can be any type or combination of sensors or devices suitable to measure or record the human energy field as vibrations, such as electromagnetic field sensors, SQUID magnetometer which is capable of detecting tiny biomagnetic fields associated with physiological activities in the body, Kirlian and enhanced computer photography, etc. Input means 8 can be any suitable pointing device, such as a Personal Computer (PC) mouse. IPU 3 can be any suitable computational device, such as a PC, a laptop, a mobile computing device and the like, or it can be a dedicated computational device provided with dedicated software for performing the required analysis and processing, as aforementioned hereinabove.

IPU 3 is connected to or comprises memory means (not shown) for storing relevant information and the software programs required for calculating, identifying and categorizing each subject according to the method of the present invention, such as:
a program for determining a consciousness scale, wherein in each value on that consciousness scale is a logarithmic value representing a frequency; a program for determining the four personality elements Trust (T), Achievement (A), Pleasure (P) and Empire (E);
a program for setting the ranges of each of the four elements on the consciousness scale; the dominate element, the stabilizer element and the transformational element; a program for determining the pairs of a dominate element and a stabilizer element (i.e., E, A and P, T);
a program for determining three energy routes for categorizing each subject into one of three energy routes by defining 12 personality types according to combinations of the four elements with the three energy routes;
a program for providing a set of question to each subject for categorizing each subject into one of the 12 personality types, and other required information and processing as aforementioned hereinabove; and
a program for calculating the difference between the subjective response and the objective response of a subject, by comparing the subjective response of the subject in correlations with the objective response of that subject, thereby determining the consciousness level of the subject, wherein while the difference between the subject response and the correlated objective response is relatively high then the consciousness level of the subject is defined as relatively low, and vice versa.

System 10 can be implemented within any suitable PC system, notebook, cellular phone, hand held device, and the like.

According to one embodiment of the invention, the system of the present invention is implemented over a network which incorporates server and clients. In such implementation, the software programs (or at least part of them) and one or more set of questions are stored and operated from a remote server (e.g., by processes running on the server side). For example, the server can be a web server using standard protocols such as TCP/IP, UDP, HTTP, etc. and the clients can be applications running on computers or other terminal units (e.g., PC, laptop, notebook, tablet PC, various kinds of mobile devices such as smart phones, iPhone, etc). The applications can be either dedicated User Interface application or a dedicated website suitable to interface with the web server.

In such embodiment, the data input device(s) (i.e., input means 8) and the one or more sensing units 6 are connected or part of the computer or other terminal units. Accordingly, the data input device(s) is used for collecting data which represents the subjective response provided by the subject to an introduced set of questions (i.e., which are used for determining the personality type of the subject), and the sensing units 6 are used for measuring the energy field of the subject while the subject answers to each question (wherein the measured energy field represents the objective response of the subject to the set of questions). The collected data and the data representing the measured energy field can be sent to the web server in order to perform the required calculation and analysis. Alternatively, at least part of the calculation and analysis can be performed locally by utilizing the terminal unit resources (depend on the terminal unit resources).

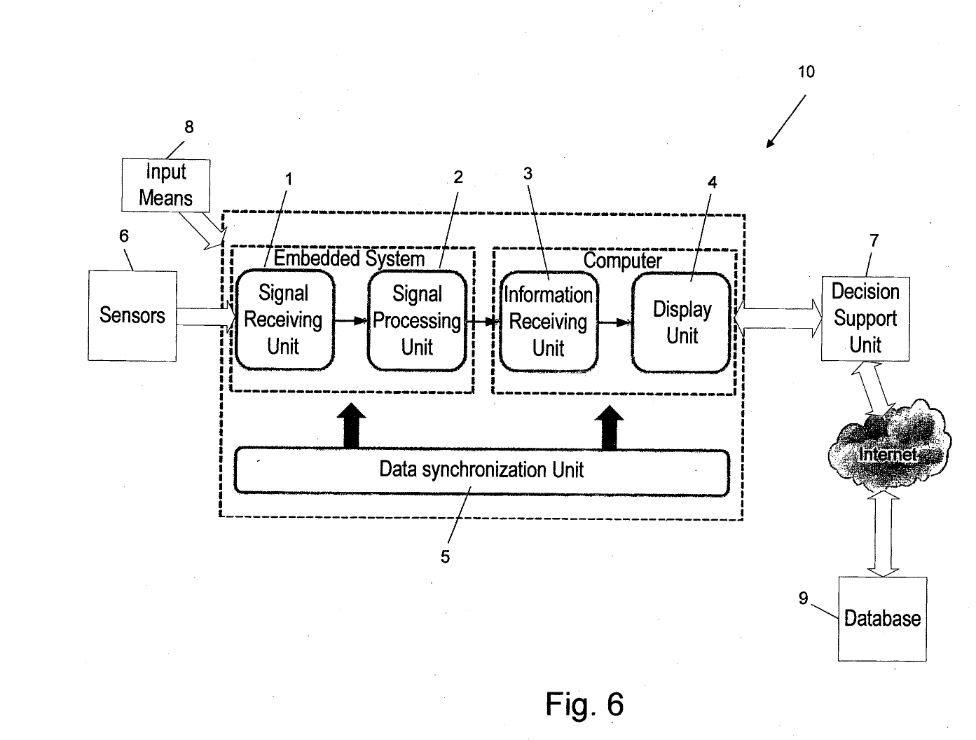

According to an embodiment of the invention, the system further comprises one or more treatment database(s) for storing data related to different ways of treatment to provide to the subjects. The treatment database can be either located locally in relation to the system 10 or remotely to system 10. In case the treatment database is located remotely to system 10, then the treatment database can be accessed via the Internet or via other network protocols. For example, FIG. 6 schematically illustrates a treatment database 9 which is remotely connected to system 10. In this figure, the database 9 communicates, via the Internet, with decision support unit 7.

According to one embodiment of the invention, the treatment provided to the subject via the treatment database is provided in real-time while the subjected is being tested by system 10, thereby allowing system 10 to provide an adaptive treatment to the tested subject according to the progress of the subject during the test.

The system of the present invention can be used as a decision making support system, which universally diagnoses of human behaviors and gives a diagnosis and recommendations to the subject. The diagnosis and recommendations are based on analyzing the difference between the subjective response and the correlated objective response of the subject and on the identified personality elements of the subject. According to an embodiment of the invention, whenever the difference between the subjective response and their correlated objective response is relatively high then generally the consciousness level of the subject is relatively low, and whenever the difference between the subjective response and their correlated objective response is relatively low then generally the consciousness level of the subject is relatively high.

Such recommendations provide the subject with a preferred way of act according to the system's diagnosis (e.g., in business decisions). Alternatively, the recommendation provides the subject with treatment suggestions which may shorten the way to raise the level of consciousness of that subject.

The system of the present invention used to detect the level of human consciousness, using calibrated values with a logarithmic sequence, as aforementioned hereinabove. The device of the present invention is also a code decoder of the individual transformation required for each person who using the system to increase his own LOC.

The system detects or identifies the basic components that determine the level of consciousness: the dominant, the stabilizer and the transformational. Where, transformational, is the key for maximizing human capabilities in all aspects of life. The TAPE model suggested by the present invention is a decoder, or a universal code of personality diagnosis, as all the criteria which are used by known methods which are used to diagnose personality, or discover human types, can be “dressed” on the TAPE model. As this TAPE model simply finds the strongest element in personality of a subject type. The TAPE model allows identifying the motive inherent in every person and starting to move in the shortest route, from a state of power, into state of strength (i.e., a higher LOC).

The technique to identify aspects of each subject is accompanied by a written test and a physiologically test that identify the strengths and weaknesses of the subject as well as the subject's personality type. The identification is done by diagnostic and detection of the subject's objective response based on the sense or response of the subject's body to the physiologic test(s), in correlation with the subject's subjectivity answers to the written test. Based on the TAPE model the system detects the subject's level of consciousness and his personality type.

According to an embodiment of the invention, system 10 is provided with two type of sensing units, a human energy field detection unit (e.g., Kirlian photography equipment) and a physiological body response sensing unit (e.g., Applied Kinesiology to sting equipment, such as the “Touchstone” by Vitascans Inc. or other electronic muscles or energy tester, such as GSR, EMG, EEG, etc). Such a muscle tester taps into the subject body's subconscious to detect how the subject really feels about a particular emotional issue or personal interaction (e.g., are you happy?

And the answer usually should be “Yes” or “No”). Accordingly, the set of question (i.e., the written test) is based on yes/no answers (i.e., “1” or “0”). In this embodiment, the system (e.g., the electronic muscles tester) should be calibrated for each individual subject, in order to determine a threshold level for “Yes” or “No” response of the subject's body (e.g., the strength of finger pressure readings or muscle response might be different from one subject to another). The system can be calibrated by introducing the subject at the beginning of the test verifying questions, such as “is your name John?”, “do you live in New-York?”, and the like. Optionally, the calibration can be performed by other sensing means, such as the Kirlian photography equipment that provides data which represent the energy field of the subject.

Based on the TAPE code and the analyzing of the objective and subjective responses of the subject, the system of the present invention detects the subject's level of consciousness and the personality type of the subject. Accordingly the transformational element of the subject is identified. The system also provides indication regarding “how far” is the distance between the objective self and the subjective self of the subject (i.e., the difference between the TAPE-O and TAPE-S of a person).

For example, a subject that responses to different type of questions that directed to the subject of “fear of taking risks in life”, and most of the subjective responses of that subject to that kind of questions are “No” (i.e., not afraid of taking risks), but the corresponding objective responses measured as “Yes”, then the difference between the objective and the subjective response indicates that this person should be positioned on the level of consciousness axis around the value 100 (i.e., Fear) on the “Trust” channel.

Furthermore, based on the provided tests to the subject, the set of questions may also include questions that are directed to estimate the hidden potential within a subject (i.e., the TAPE-M which represents the highest LOC a subject may reach), this can be achieved by using questions that are based on common theories, typologies or techniques which are used for estimation such as, numerology, Lüscher color test, Enneagram theory, Gann's square of nine, etc. Knowing the TAPE-M of a subject (the target which the subject should aim to) together with the identified transformational element can provide the subject with recommendations that may aid the subject to increase it's LOC with minimal effort.

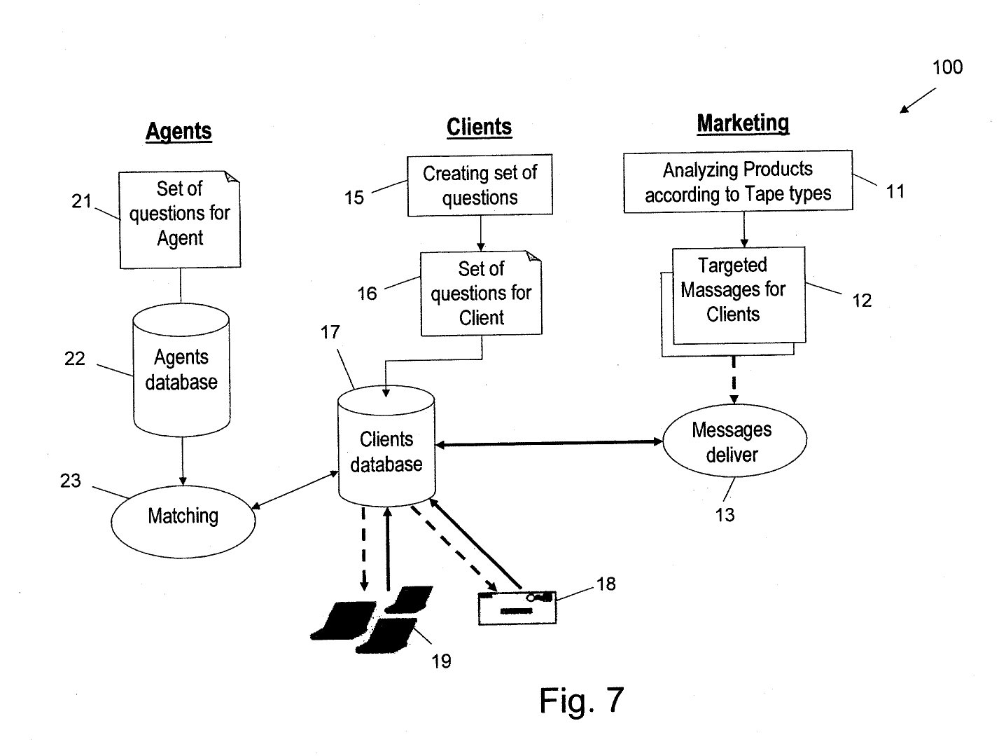

According to another embodiment of the invention, the system can be used for matching between persons, e.g., for business and marketing propose, for matching between clients and targeted marketing messages or between clients and company agents, such as salesman, for matching between student and teacher, for matching between a man and a woman, etc. FIG. 7 schematically illustrates an example for such a system 100 for matching between clients and agents and products based on the TAPE code personality type diagnosis of the present invention.

System 100 may comprises the following matching channels, the clients' channel, the marketing channel, and/or the agents' channel. The clients channel comprises the following steps:
Building (block 15) one or more sets of questions for identifying the dominant element of each client. Optionally, each set of questions is directed to different type of clients population (e.g., demographic location, age, and the like);
Providing (block 16) set of a questions to each client, via any applicable communication method, such as while a client visit in a shop, a webpage, etc., and according to clients answers to the set of question, categorizing each client profile according to at least the dominant element; and
Storing each categorized client in a clients' database 17.

The marketing channel comprises the following steps:
Analyzing (block 11) the way of introducing the same product according to each TAPE personality type as defined by the method of the present invention;
Generating (block 12) targeted messages for each personality type; and
Matching between generated targeted messages and the clients according to their stored categorized profile and delivering (block 13) the generated targeted messages to these clients. For example, via SMS, e-mail 18, advertisement on a webpage 19, etc.

The agents channel comprising the following steps:
Providing a set of questions (block 21) to each agents for categorizing each agent according to the 12 personality TAPE types;
Storing each categorized agent in an agents' database 22; and
Matching between agents (stored in database 22) and clients (stored in database 17) having the same dominate personality type, thereby increasing the chances for “closing” a transaction. Of course, this is based on the assumptions that persons having matching personality type code can better interact.

Therefore, if an agent can recognize the dominant element of a client, then the agent can scientifically increase the chances of “closing” (i.e., succeeding in executing a transaction with that client, e.g., to sell that client a product or service). This can be done by identifying at least one of the following four elements Mo, Fr, Im, Pr:
(Mo)—Identifying what parameter(s) might drive (or motivate) the client to perform an action (e.g., drive or encourage the client to buy a specific product) as derived from the dominant element of the client;
(Fr)—Identifying and matching the communication frequency with the client according the client's dominant and stabilizer elements;
(Im)—Showing to the client an imagined “future reality” with the product/service, using a simulation which is aimed to the dominant element of the client and the parameter(s) that might drive him, in order to allow the client to “see in his mind”, in a positive manner, his future use with the product; and
(Pr)—Emphasizing the client's profit, by introducing the society's comparative advantage based on his motive (from the dominant element), thereby raising the client's level of satisfaction to be greater than the cost he need to pay for the product.

As all these four elements (Mo, Fr, Im, and Pr) are directly related to each other, and a function of them (all together or at least part of them) enhance or increase the chances in succeeding with the “closing” (e.g., f(Mo, Fr, Im, Pr)=“closing”).

For example, the parameter(s) that might drive or motivate the client to by a specific product can be as follows (assuming that such parameters which are aimed to the dominant element may better drive or motivate the client to purchase the specific product):
if the dominant element of the client is Trust (i.e., “T”) then the agent should emphasis to the client one or more parameters which are related to the trust aspects of s specific product, such as safety of the specific product, manufacturer guarantee, the product is manufactured by a leading company in the field, etc.;
If the dominant element is Achievement (i.e., “A”) then the agent should emphasis to the client one or more parameters which are related to the achievement aspects, such as what is the profit for the client from the product, the benefit from the product, special low price, etc.;
If the dominant element is Pleasure (i.e., “P”) then the agent should emphasis to the client one or more parameters which are related to the pleasure aspects, such as the beauty of the product, pleasant shopping experience, etc.; and
If the dominant element is Empire (i.e., “E”) then the agent should emphasis to the client one or more parameters which are related to the client ego, such as whether the product is exclusive, what the product will do for the client, etc.

As aforementioned hereinabove, the technique to identify aspects of each subject is accompanied by a written test and a physiologically test that identify the strengths and weaknesses of the subject as well as the subject's personality type. The identification is done by diagnostic and detection of the subject's objective response based on the sense or response of the subject's body to the physiologic test, in correlation with the subject's subjectivity response to the written test. Based on the TAPE model the system detects the subject's level of consciousness. The system is a universal diagnosis for the human types and their level of consciousness. In some cases for matching purpose, the written test (i.e., the set of questions) can be used to detect the dominate element of the TAPE personality types of a subject without any physiological test. For example, this may occur when one of the matching sides is a client remotely located from the system, thus the client can answer only to a written test (e.g., via an Internet website).

According to some embodiments of the present invention, the technique to identify aspects of each subject can be obtained by using a voice spectral analysis (i.e., using human Bioacoustics test instead of the written test) that can be used to identify the strengths and weaknesses of the subject as well as the subject's personality type. Alternatively, such technique can also be used together with other physiologically test as described hereinbefore.

Human Bioacoustics refers to cross-disciplinary techniques that combines biology and acoustics. It includes voice spectral analysis that models the frequencies and architecture of human vocalizations to identify the innate mathematical templates found within the various system of the human body. These non-invasive techniques are being advanced to the extent that a computerized Vocal Profile, using a system of Frequency Equivalents, can be used to accurately quantify, organize, interpret, define, and extrapolate biometric information from the human voice. This information, in turn, provides the opportunity to obtain the four elements of the TAPE code.

The system of the present invention can be based on any suitable voice spectral analysis techniques. For example, such as the ones disclosed in “OpenEAR—Introducing the Munich open-source emotion and affect recognition toolkit”, by F. Eyben, et al., pages 1-6, Affective Computing and Intelligent Interaction and Workshops, 2009. ACII 2009. 3rd International Conference on, in the “Speech emotion recognition based on HMM and SVM” by Yi-Lin Lin et al., vol. 8, pages 4898-4901, Machine Learning and Cybernetics, 2005. Proceedings of 2005 International Conference on, in the “Recognition of hesitations in spontaneous speech” by D. O'Shaughnessy, vol. 1, pages 521-524, Acoustics, Speech, and Signal Processing, 1992, ICASSP-92, 1992 IEEE International Conference on.

The voice spectral analysis can be done over telephone lines and with standard commercially available microphones. Based on such technologies and capabilities, the aforementioned “closing” between a client and an agent can be performed via a telephone conversation as the client's voice parameters can be analyzed over a telephone line.

The following Table (3) shows an example for setting the TAPE code according to several voice related parameters of a subject:

TABLE 3

| | T | A | P | E |
|---|---|---|---|---|
| Speech Intensity | 4 | 2 | 3 | 1 |
| Speech Pace | 2 | 4 | 3 | 1 |
| Speech Tone Recognition | 4 | 1 | 3 | 2 |
| Speech Monotony Recognition | 1 | 4 | 3 | 2 |
| Speech Mobility Recognition | 4 | 1 | 2 | 3 |
| Hyper Speaker Enthusiasm Recognition | 4 | 1 | 2 | 3 |
| The fastest waiting time for a response | 4 | 1 | 3 | 2 |
| form the subject | | | | |
| Speaker Hesitation recognition | 2 | 3 | 1 | 4 |
| First sentence spoken in determination | 2 | 3 | 4 | 1 |
| manner | | | | |
| Interference while the person on the | 4 | 1 | 3 | 2 |
| other side (of the phone) still talking | | | | |

For example, in the table 3 the T, A, P and E values are set from 1 (which represents the higher voice related value) to 4 (which represents the lower voice related value).

While some embodiments of the invention have been described by way of illustration, it will be apparent that the invention can be carried into practice with many modifications, variations and adaptations, and with the use of numerous equivalents or alternative solutions that are within the scope of persons skilled in the art, without departing from the spirit of the invention or exceeding the scope of the claims.

1-33. (canceled)

34. A method for detecting a universal consciousness personality type code of a subject, comprising:
a) determining four root elements T, A, P and E for representing the DNA-like individual personality code of said subject, and setting the ranges of each of said elements on a consciousness scale, wherein the DNA-like individual personality code for each subject is defined by three elements among said four elements, one as a dominate element, one as a stabilizer element and one as a transformational element, wherein the relations between the dominate element, the stabilizer element and the transformational element is such that a relatively slight change in the transformational element will result in the largest change in the dominant and the stabilizer elements, thereby allowing said subject to move up from the current level of consciousness to a higher level with minimum effort, wherein the ratios between said three elements are determined as follows: the dominate element reflects 50% of the subject's personally, the stabilizer element reflects 30% and the transformational element reflects 20%;
b) determining three energy routes for categorizing each subject into one of said three energy routes, wherein said three energy routes are defined as Dominant energy people, Constant energy people and Varied energy people;
c) determining two possible pairs of dominate and stabilizer elements, wherein one pair of elements is E and A (or A and E) which represents a person who motivates with respect to an external energy source and the other pair of element is P and T (or T and P) which represents a person who motivates with respect to an internal energy source, wherein each pair of element is determined according to the dominant element which can be T or A or P or E;
d) defining 12 basic personality type codes according to combinations of the four root elements together with said three energy routes, thus the dominate element of each subject together with the corresponding energy route is used for categorizing each subject into one of said 12 basic personality types, and wherein said 12 basic personality type codes are ordered in a circular manner in a corresponding TAPE code diagram, wherein the route of said internal energy source type of person is represented as a clockwise movement in said TAPE code diagram and the route of said eternal energy source type of person is represented as a counterclockwise movement in said TAPE code diagram;
e) detecting the dominate element and the energy route of said subject by obtaining the objective feedback and optionally by also obtaining the subjective response of said subject to one or more tests, wherein said objective feedback represent the responses of the subject's body to said one or more tests;
f) identifying the stabilizer element according to said detected dominant element as determined by one of said pairs of dominate and stabilizer elements; and
g) defining the transformational element of said subject according to said detected dominant element and the direction of the route of said internal or external energy source, as a results the dominate and transformational elements can be A and T or E and P, in such a manner that if the dominate element is A than the transformational element is T or vice versa, and if the dominate element is E than the transformational element is P or vice versa.

35. A method according to claim 34, further comprising measuring the consciousness level of the subject, wherein the one or more tests are a set of questions introduced for said subject, by performing the steps of:
a) measuring the energy field of said subject by one or more sensing units while said subject answers to each introduced question, wherein said measured energy field represent the objective feedback of said subject to each of the introduced question while the answers of said subject to each question represent the subjective response;
b) detecting the dominate element of said subject from said measured energy field and accordingly detecting the TAPE code of said subject which includes also the stabilizer and the transformational elements; and
c) comparing the subjective response of said subject (i.e., TAPE-S) with the measured objective feedback of said subject (i.e., TAPE-O) with respect to said detected TAPE code of said subject (i.e., TAPE-M) for determining the consciousness level of said subject, wherein while the difference (i.e., the delta) between said subjective response and said measured objective response is relatively high, then the consciousness level of said subject is determined as relatively low on the consciousness scale, and vice versa.

36. A method according to claim 35, wherein the objective feedback, the measuring of the energy field of the subject and/or the dominate element are obtained by using a voice spectral analysis of the subject's vocalizations.

37. A method according to claim 35, further comprises providing a decision support unit for providing recommendations to the subject according to measured consciousness level and/or providing treatments or treatment suggestions for the subject according to the transformational element of said subject, according to difference between the objective feedback and the subjective response of said subject and/or the ratios between the three elements.

38. A method according to claim 34, further comprising presenting the 12 personality type codes on the nodes of a Merkava model for calculating the shortest route from the node representing the identified subject's personality to central node of said Merkava model, wherein said shortest route represents the minimum effort required from said subject in order to increase the level of consciousness of said subject, wherein the transformational element of each personality type code is found in the personality type code adjacent to it on said Merkava model with respect to the direction of the energy route, whether it defined as clockwise or counterclockwise.

39. A method according to claim 34, further comprising matching between at least two subjects having at least the same dominate elements, by providing at least one database for storing the categorized subjects, wherein the matching comprises a clients' channel, a marketing channel, an agents' channel or any other matching cases.

40. A method according to claim 39, wherein the clients channel comprising the steps of:
a) identifying the dominant element of each client by a sensing unit;
b) optionally, providing a set of a questions to each client, via any applicable communication method, and according to the objective feedback of said client to said set of question or the objective feedback and the subjective response of said client to said set of question, categorizing each client profile according to at least the identified dominant element of each client; and
c) storing each categorized client in a clients' database;
wherein the marketing channel comprises the steps of:
d) analyzing the way of introducing the same product according to each defined personality type code;
e) generating targeted messages for each personality type code; and
f) matching between generated targeted messages and the clients according to their stored categorized profile and delivering said generated targeted messages to said clients; and
wherein the agents channel comprising the steps of:
g) providing a set of questions to each agents for categorizing each agent according to the 12 personality TAPE types;
h) storing each categorized agent in an agents' database; and
i) matching between profiles of agents stored in said agents' database and clients stored in the clients database having the same dominate personality type, thereby increasing the chances for “closing” a transaction.

41. A method according to claim 34, further comprising identifying an element which drives or motivates the subject to perform an action, wherein said element is derived from the dominant element of said client.

42. A method according to claim 39, wherein the matching can be done by identifying at least one of the following four elements Mo, Fr, Im, Pr, wherein all of said four elements are directly related to each other, while a function of them enhances or increases the chances in succeeding with “closing” a transaction.

43. A system for detecting the personality type code of a subject, comprising:
a) an information processing unit suitable to execute software code for performing the following tasks:
I. detecting one or more parameters related to the personality type of said subject, wherein said software code is used for determining a consciousness scale;
II. determining four root personality parameters T, A, P and E for representing the individual personality type code of said subject, and
III. setting the ranges of each of said parameters on said consciousness scale, wherein each three of said four root personality parameters represent the dominate element, the stabilizer element and the transformational element for each individual subject, wherein said dominate element can be detected according to the objective response of said subject to one or more tests, said stabilizer element can be defined according to the said dominant element and said transformational element can be determined according to said dominant and stabilizer element, wherein said transformational element can be used to allow a person to improve its individual level of consciousness with minimum effort;
b) a data input device for collecting data representing voice parameters of said subject, wherein said voice parameters represent the objective response of said subject to said one or more tests; and
c) a sensing unit for obtaining the objective feedback of the subject in order to detect the dominate element of said subject.

44. A system according to claim 43, in which the sensing unit is a voice spectral analysis means for extrapolating information from the voice of the subject for detecting the dominate element in the personality consciousness type code of the subject.

45. A system for measuring the consciousness level of a subject, comprising:
a) data input device for collecting data representing the subjective response provided by said subject to an introduced set of questions, wherein said set of questions is used for determining the personality type of said subject;
b) one or more sensing units for measuring the energy field of said subject while said subject response to each of said questions, wherein said measured energy field represents the objective feedback of said subject to each of said questions;
c) memory unit for storing data representing all the relevant parameters of said subjective and objective responses;
d) information processing unit suitable to execute software code for detecting the personality type of said subject which includes the dominate element, the stabilizer element and transformational element, wherein said elements are detected by analyzing the response of said subject to each question of said set of questions in correlations with the corresponding measured energy field, thereby determining the consciousness level of said subject wherein while the difference between said subject response and said correlated objective response is relatively high then the consciousness level of said subject is relatively low, and vice versa, wherein reducing the difference on the level of consciousness scale between the objective response and the subjective response of said subject will increase the level of consciousness of said subject, and in order to effectively reduce this difference, the transformational element is provided, so as to aid said subject to improve the individual level of consciousness with minimum effort, in order to allow a person to improve its individual level of consciousness it is required to reduce the difference on the level of consciousness scale between the objective feedback and the subjective response of that person to minimum, wherein the values of each element of the personality type code on the consciousness scale are calculated and compared with an expected usual ratios between the elements of a person that is determined as follows: the dominate element reflects 50% of the personally, the stabilizer element reflects 30% and the transformational element reflects 20%; and
e) data synchronization unit for synchronizing between the data representing each of the response provided by said subject to said set of questions and the corresponding measured vibrations.

46. A system according to claim 45, in which the software code includes:
a) a program for determining a consciousness scale, wherein in each value on said scale is a logarithmic value which represents a frequency;
b) a program for determining four personality parameters T, A, P and E for representing the individual personality type code of a subject, and for setting the ranges of each of said parameters on said consciousness scale, wherein the personality type code for each subject comprises three parameters among said four parameters, the first is a dominate element, the second is a stabilizer element and the third is a transformational element;
c) a program for determining two pairs of said parameters, each pair comprises a dominate element and a stabilizer element, wherein the first pair of elements is E and A (or A and E), and the second pair of element is P and T (or T and P);
d) a program for determining three energy routes for categorizing each subject into one of said three energy routes by defining 12 personality types according to combinations of said four elements with said three energy routes, wherein the energy route of said subject is identified according to the objective feedback of said subject, the subjective response of said subject or according to the comparison between said objective feedback and subjective response;
e) a program for identifying the stabilizer element of said subject according to the pair of elements in which said dominate element is found, and for identifying the transformational element according the identified energy route of said subject, whether it internal or external energy source; and
f) a program for calculating the ratios between the elements of the person

47. A system according to claim 45, further comprises a display unit for introducing the set of questions to the subject and for displaying the determined consciousness level of said subject providing recommendations to the subject according to the determined consciousness level of said subject, for allowing moving “up” in the consciousness scale with minimal effort according to the transformational element.

48. A system according to claim 45, further comprises a decision support unit for providing recommendations to the subject according to the identified transformational element and the energy route of said subject and/or for providing recommendations to the subject according to the determined consciousness level of said subject with respect to the usual ratio.

49. A system according to claim 45, in which the one or more sensing units are selected from the group consisting of: GSR, EMG, EEG, electromagnetic field sensors, Kirlian photography, instrumentation used to record the human energy field, a voice spectral analysis means or any combination thereof.

50. A system according to claim 45, further comprising treatment database for storing data related to different ways of treatments either data that obtained in real time during the treatment or previously stored data, wherein said treatment database can be accessed locally or remotely via the Internet or other network protocols, wherein data from the treatment database can be provided in real-time while said subjected is being tested by said system, thereby allowing said system to provide an adaptive treatment to said subject according to the progress of said subject during said test.

51. A system according to claim 45, further comprising voice capturing and analyzing means for detecting the dominate element in the personality consciousness type code of the subject.

52. A system according to claim 45, further comprising a decision support unit for providing recommendations to a person according to the results of the calculated values of each element of the personality type code on the consciousness scale and their comparison with the expected usual ratios between the elements of the person, such that said person will receive the relevant treatment in order to reach the expected usual ratios between said elements.
